(19) United States
(12) Patent Application Publication
(10) Pub. No.: US 2009/0137941 A1
(43) Pub. Date: May 28, 2009

(54) METHOD AND APPARATUS FOR ACOUSTICALLY ENHANCED REMOVAL OF BUBBLES FROM A FLUID

(75) Inventors: John E. Lynch, Williamsburg, VA (US); Christopher S. Domack, Carrollton, VA (US)

Correspondence Address: NIXON & VANDERHYE, PC, 901 NORTH GLEBE ROAD, 11TH FLOOR, ARLINGTON, VA, 22203, US

(73) Assignee: Luna Innovations Incorporation, Roanoke, VA (US)

(21) Appl. No.: 12/129,985

(22) Filed: May 30, 2008

Related U.S. Application Data
(60) Provisional application No. 60/924,962, filed on Jun. 6, 2007.

(51) Int. Cl. A61M 1/36; B01D 19/00

(52) U.S. Cl. 604/6.11; 96/175; 95/30

(57) ABSTRACT

A vessel for removing bubbles from a fluid is provided. The vessel includes a fluid inlet port for receiving the fluid, and a bubble outlet port for removing bubbles in the fluid from the vessel. An ultrasonic transducer is mounted in the vessel and transmits an ultrasonic beam through the received fluid to move bubbles in the fluid towards the bubble outlet port. A fluid outlet port outputs the fluid insonified by the ultrasonic beam. An ultrasonic reflector mounted near the bubble outlet port reflects the ultrasonic beam away from the fluid outlet port to reduce or prevent reflection of the ultrasonic beam off an interior surface in the vessel directed towards the fluid outlet port.

### RELATED APPLICATION

This application claims the priority and benefit of U.S. Provisional patent application 60/924,962, filed Jun. 6, 2007, which is incorporated herein by reference in its entirety.

### TECHNICAL FIELD

The technology relates to removing of bubbles of gas from a fluid. One non-limiting example application is to the removal of gaseous emboli from blood circulated in an extracorporeal blood circuit, such as in a heart-lung machine or in a dialysis machine.

### BACKGROUND

An embolus is a structure that travels through the bloodstream, lodges in a blood vessel and blocks it. Examples of emboli are a detached blood clot, a clump of bacteria, foreign material, and air bubbles. In surgical operations, heart surgery in particular, there is a relationship between increased number of emboli present in blood delivered to the brain, i.e., the embolic load delivered to the brain, and neurocognitive deficits. As a result, arterial line filters may be employed in an extracorporeal blood (CPB) circuit to filter out emboli from the blood circulating in the circuit. Unfortunately, arterial line filters include pores large enough, e.g., 28 to 40·10⁻⁶ m (28 to 40 μm), to allow smaller emboli to pass through, and larger air and fat emboli also pass through and enter the circulation downstream to the filter whenever their load is high. Significantly, microbubbles that pass through the arterial line filter join together and become large bubbles potentially causing harm to the patient. This problem is particularly severe in low-prime bypass circuits, so that despite their advantages (e.g., lower prime volume results in higher hematocrit values, less systemic inflammation, less platelet activation, and better oxygen delivery to the patient), low-prime bypass circuits do not purge venous air from the system as well as traditional bypass circuits.

So there is a need for a better method for removing gaseous emboli from the bypass circuit prior to returning blood to the patient. The inventors considered various ways to remove gaseous emboli from the blood. One way is to increase the amount of fluid in a bubble removal vessel in the CBP circuit, for example, by increasing the cross-sectional area of a bubble removal vessel. This increased cross-section effectively slows down blood flow in the CBP bubble removal vessel which makes bubbles easier to trap and remove. A wider cross-sectional area also creates a smaller pressure drop from the inlet to the outlet so that the buoyant force of the bubble may be used to separate air bubbles from blood. Similarly, CBP bubble removal components may be made taller to give the bubbles more time to overcome the flow velocity and rise into a gas purge outlet in the vessel. However, there is a limit to the size of the vessel that may be used during bypass—as larger vessels require greater dilution of blood with a priming solution and increased use of transfused blood. It would be desirable to remove microbubbles from a bypass circuit while reducing the size of the circuit, thus leading to reduced dependence on transfused blood during cardiopulmonary bypass surgery.

Another way to remove air from blood is by causing the blood to move in a swirling flow so that air bubbles are pulled to the center of the swirl as in a centrifuge. Alternatively, the pressure within a CBP bubble removal component may be controlled to discourage formation of gas bubbles. Both techniques can be effective in removing larger bubbles, but do not remove microbubbles which are more difficult to remove from flowing blood due to their reduced buoyant force.

Ultrasound waves, which have acoustic radiation force, can used to actively remove bubbles. An ultrasonic wave carries momentum that is transferred to a particle, e.g., an air bubble, upon reflection or absorption of the sound wave.

What is needed is technology that can remove both large bubbles and microbubbles.

### SUMMARY

The ultrasonic bubble removal technology described here removes bubbles including very small microbubbles from a fluid. In addition to ultrasonic bubble removal, additional bubble removal mechanisms are used to enhance the reliability of bubble removal. These additional bubble removal mechanisms also ensure that ultrasonic power levels can be kept below established safety guidelines in sensitive applications like CBP gaseous emboli removal.

A vessel for removing bubbles from a fluid is provided. The vessel includes a fluid inlet port for receiving the fluid, and a bubble outlet port for removing bubbles in the fluid from the vessel. An ultrasonic transducer is mounted in the vessel and transmits an ultrasonic beam through the received fluid to move bubbles in the fluid towards the bubble outlet port. A fluid outlet port outputs the fluid insonified by the ultrasonic beam. An ultrasonic reflector mounted near the bubble outlet port reflects the ultrasonic beam away from the fluid outlet port to reduce or prevent reflection of the ultrasonic beam off an interior surface in the vessel directed towards the fluid outlet port. Preferably, the reflector is mounted to reflect the ultrasonic beam away from the fluid outlet port but also in way that increases the amount of acoustic radiation force directed towards the bubble outlet port.

The vessel may include a barrier having a first barrier portion that separates the fluid inlet port and the fluid outlet port. An opening in the first barrier portion permits the ultrasonic beam to radiate fluid received from the fluid inlet port and allows the received fluid from the fluid inlet port to reach the fluid outlet port. In a non-limiting preferred embodiment, the opening is sized to at least substantially match a width of the ultrasonic beam, and the reflector is positioned to reflect the ultrasonic beam away from the opening. The barrier includes a second barrier portion at a sufficient angle to the first barrier portion to create the opening, the second barrier portion extending past the fluid outlet port. In a non-limiting preferred embodiment, the first barrier portion is substantially perpendicular to a sidewall of the vessel, and the first barrier portion and the second barrier portion are substantially perpendicular. The opening may be circular and the second barrier portion cylindrically-shaped. In an alternative example configuration, the second barrier portion includes concentric cylindrically-shaped surfaces.

The vessel preferably includes an acoustically transparent material separating the ultrasonic transducer from the fluid inlet port and the fluid outlet port. A cooling fluid inlet receives cooling fluid that removes heat from the vessel caused by the ultrasonic transducer, and a cooling fluid outlet removes the cooling fluid from the vessel. The acoustically transparent material prevents the cooling fluid from contacting the received fluid. In one example configuration, the acoustically transparent material is shaped to adjust the ultrasound beam so that a profile of the ultrasound beam approximates the dimensions of the opening in the barrier. The acoustically transparent material defines an ultrasonic standoff region in the vessel between the acoustically transparent material and the ultrasonic transducer. In a non-limiting preferred embodiment, the length of the ultrasonic standoff region substantially matches a near-field/far-field transition of the ultrasonic beam where the ultrasonic wave is at a maximum amplitude.

Different non-limiting configurations of the vessel are described. For example, the ultrasound beam, the opening in the barrier, and the acoustic reflector may be substantially aligned along a same axis. The bubble outlet port may be substantially aligned along the same axis, or it may be offset from and not aligned with the same axis. The ultrasonic transducer may be shaped to focus the energy of the ultrasonic beam through the opening. If the vessel is cylindrically-shaped, the fluid inlet port and the fluid outlet port are preferably oriented substantially tangential to a cylindrical surface of the vessel to produce a swirling flow of the received fluid in the vessel that forces bubbles to the center of the vessel in line with the opening and coalesces smaller ones of the bubbles into larger bubbles. The bubble outlet is preferably located at or near a highest point of the vessel when the vessel is mounted for operation.

In a non-limiting example embodiment, the vessel includes a porous mesh positioned in a direction that is substantially parallel to the first barrier portion and covers the opening. The porous mesh mechanically traps bubbles larger than a pore size of the porous mesh, and the ultrasonic beam forces the bubbles towards the bubble outlet port. Alternatively, porous mesh may be positioned in a direction having a substantial angle with the first barrier portion between the fluid inlet port and the opening and between the opening and the fluid outlet port. The angled mesh provides greater surface area for trapping bubbles and reduces the possibility of clogging the mesh with particles that could obstruct flow.

One example advantageous application is a system for removing gaseous emboli from blood. The system includes a blood circuit receiving blood from a patient. A pump coupled to the blood circuit pumps the blood through the blood circuit. A vessel coupled to the blood circuit removes gaseous emboli from blood. The vessel includes a blood inlet port for receiving the blood, and an emboli outlet port for removing gaseous emboli in the blood from the vessel. An ultrasonic transducer mounted in the vessel that transmits an ultrasonic beam through the received fluid to move gaseous emboli in the fluid towards the gaseous emboli outlet port. A blood outlet port of the vessel outputs the blood insonified by the ultrasonic beam. An ultrasonic reflector mounted near the gaseous emboli outlet port reflects the ultrasonic beam away from the blood outlet port to reduce or prevent reflection of the ultrasonic beam off an interior surface in the vessel directed towards the blood outlet port. The reflector is mounted to reflect the ultrasonic beam away from the gaseous emboli outlet port and to increase an amount of acoustic radiation force directed upwards towards the gaseous emboli outlet port. The system includes a controller for controlling the ultrasonic transducer and the pump.

The blood circuit preferably includes a sensor for sensing gaseous emboli in the blood entering the vessel and providing sensor information to the controller for use by the controller in controlling operation of the ultrasonic transducer. Another sensor for sensing gaseous emboli in the blood exiting the vessel may also be used to detect when gaseous emboli still remains in the blood.

The vessel may be provided in variety of locations in the blood circuit. For example, the vessel may be provided in one or more of the following blood circuit components: a venous reservoir, an arterial line filter, or a bubble trap.

A method for debubbling a liquid is also described. The liquid is introduced to a vessel through a fluid inlet and flows through the vessel, preferably in a spiral path, toward a first outlet. An ultrasonic transducer within the vessel transmits an ultrasonic beam along a longitudinal axis the vessel toward the spiral path and toward a second outlet. The ultrasonic beam reflects within the vessel away from the blood outlet port to reduce or prevent reflection of the ultrasonic beam off an interior surface in the vessel directed towards the first outlet. The ultrasonic beam is also reflected away from the second outlet to increase an amount of acoustic radiation force directed upwards towards the second outlet. A stream of insonified liquid is withdrawn through the first outlet, and a stream of liquid containing entrained air bubbles is withdrawn through the second outlet.

### BRIEF DESCRIPTION OF THE DRAWINGS

FIG. 1(a) is a non-limiting example of an extracorporeal blood (CPB) circuit in which gaseous emboli are removed;

FIG. 1(b) is another non-limiting example CPB circuit in which gaseous emboli are removed;

FIG. 1(c) is another non-limiting example CPB circuit in which gaseous emboli are removed;

FIG. 2 is a front perspective view of an ultrasound-assisted debubbling apparatus in accordance with a non-limiting example embodiment;

FIG. 3 is a cross-sectional view of the ultrasound-assisted debubbling apparatus in FIG. 2;

FIG. 4 a three dimensional, perspective, cross-sectional view of the ultrasound-assisted debubbling apparatus of FIG. 2;

FIG. 5 is a top view of the ultrasound-assisted debubbling apparatus of FIG. 2;

FIG. 6 is a partial cross-sectional view of the ultrasound-assisted debubbling apparatus of FIG. 2 showing ultrasonic beam reflections;

FIG. 7 is a cross-section of the ultrasound-assisted debubbling apparatus of FIG. 2 showing a non-limiting example embodiment with a curved ultrasonic transducer for shaping the ultrasonic beam;

FIG. 8 is a cross-sectional view showing an alternative example embodiment of the debubbling apparatus in FIG. 2 showing a non-limiting example embodiment with a differently-shaped barrier separating an ultrasonic stand-off region from fluid regions in the vessel;

FIG. 9 is a cross-sectional view of the debubbling apparatus in FIG. 2 showing a non-limiting example embodiment with the barrier structure between the fluid inlet and fluid outlet ports having a concentrically-shaped portion;

FIG. 10 is a partial cross-sectional view of the debubbling apparatus in FIG. 2 showing an alternative example embodiment where the bubble outlet port is off-center;

FIG. 11 is a side view of an alternative example embodiment for delivering fluid to the debubbling apparatus shown in FIG. 2; and

FIGS. 12(a) and 12(b) are cross-sectional views of alternative example embodiments of the debubbling apparatus in FIG. 2 employing one or more porous meshes to filter bubbles.

### DETAILED DESCRIPTION

The following description sets forth specific details, such as particular embodiments, procedures, techniques, etc. for purposes of explanation and not limitation. But it will be appreciated by one skilled in the art that other embodiments may be employed apart from these specific details. In some instances, detailed descriptions of well known methods, circuits, and devices are omitted so as not obscure the description with unnecessary detail. Moreover, individual blocks are shown in some of the figures. Those skilled in the art will appreciate that the function of the controller block may be implemented using individual hardware circuits, using software programs and data, in conjunction with a suitably programmed digital microprocessor or general purpose computer, using application specific integrated circuitry (ASIC), and/or using one or more digital signal processors (DSPs).

As explained in the background, one particularly advantageous application for the technology described in this application is in the context of extra corporeal blood (CPB) circuits. However, those skilled in the art will appreciate that this is a non-limiting example application and that the technology in this case may be applied to any fluid from which any type of bubble is to be removed from the liquid. Other example applications include removing bubbles from a photographic emulsion or from certain industrial components whose performance requires a substantially bubble-free fluid. Although air bubbles are a common example, the term “bubble” includes any type of gas dissolved in or otherwise embedded in a liquid.

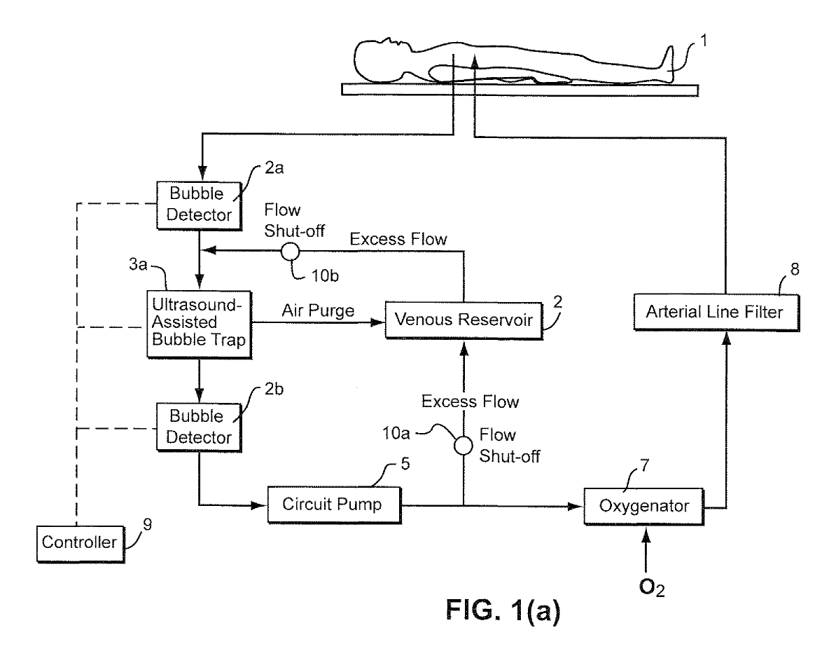

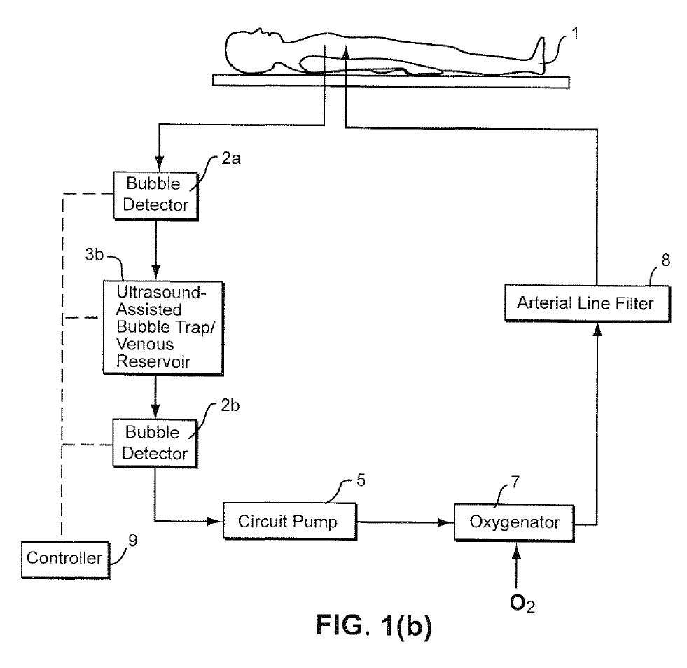

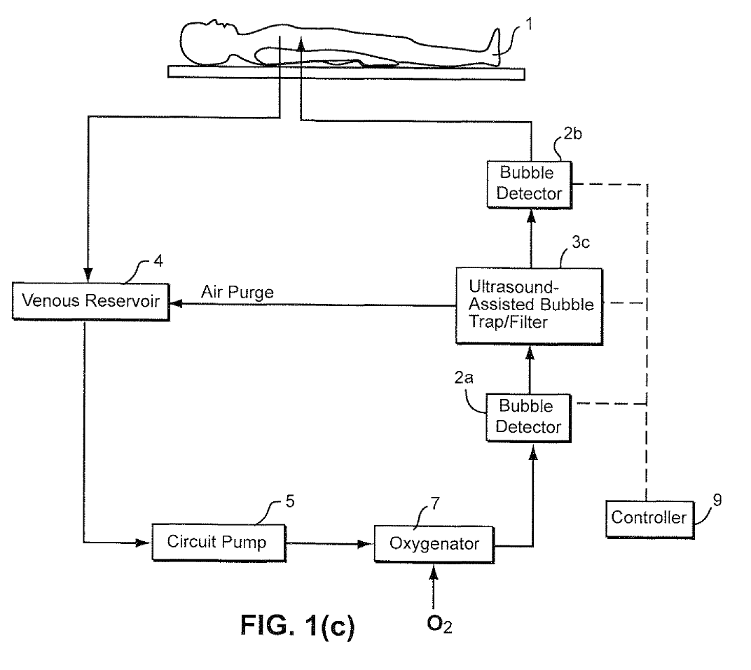

FIG. 1(a) is a non-limiting example of an extracorporeal blood (CPB) circuit in which gaseous emboli are removed. A patient 1 is shown coupled to the CPB circuit. Blood from the patient 1 is provided to a bubble detector 2a which detects the presence of bubbles in the blood and provides a signal to a controller 9. A suitable example bubble detector is an ultrasonic microemboli detector such as the EDAC® Quantifier from Luna Innovations Inc. The blood continues to an ultrasound-assisted bubble removing vessel or “trap” corresponding to a debubbling apparatus 3a. The debubbling apparatus 3a removes air bubbles and other gaseous emboli from the blood and vents them via an air purge line to a venous reservoir 2.

Blood from the debubbling apparatus 3a may be monitored by a second bubble detector 2b to determine if any bubbles remain in the blood. If bubbles are detected, the second bubble detector 2b notifies the controller 9 that bubbles remain in the blood and corrective action is taken to prevent additional bubbles from exiting the debubbling apparatus. The blood is provided through a circuit pump 5 which keeps the blood moving throughout the CPB circuit. The output fluid from the circuit pump 5 may be provided back to the venous reservoir 2 via a flow shut-off valve 10a. This shut-off valve is useful to maintain the correct volume of blood flow in the CPB circuit by removing excess blood. Blood may also be returned to the CPB circuit and to debubbling apparatus 3a from the venous reservoir via a second flow shut-off valve 10b. The flow shut-off valves may be controlled by the controller 9 or may be manually controlled.

When the flow shut-off valve 10a is closed, the blood in the circuit flows to an oxygenator 7 in which oxygen is infused into the blood. The oxygenated blood is then provided to an optional arterial line filter 8 which provides additional protection for the patient 1 from filters out gaseous and solid emboli. Details of non-limiting examples of the debubbling apparatus 3 will be described below in conjunction with subsequent figures.

The controller 9 receives information from the optional bubble detectors 2a and 2b, controls the ultrasound assisted bubble trap 3a, and may also control the shut-off valves. The controller 9 operates the ultrasound transducer in the debubbling apparatus 3 at an appropriate power level and frequency. When no bubbles are detected by bubble detector 2a or 2b, the controller 9 may optionally deactivate the debubbling apparatus 3. Alternatively, it may be desirable to operate the debubbling apparatus 3 as long as blood is flowing through the CPB circuit.

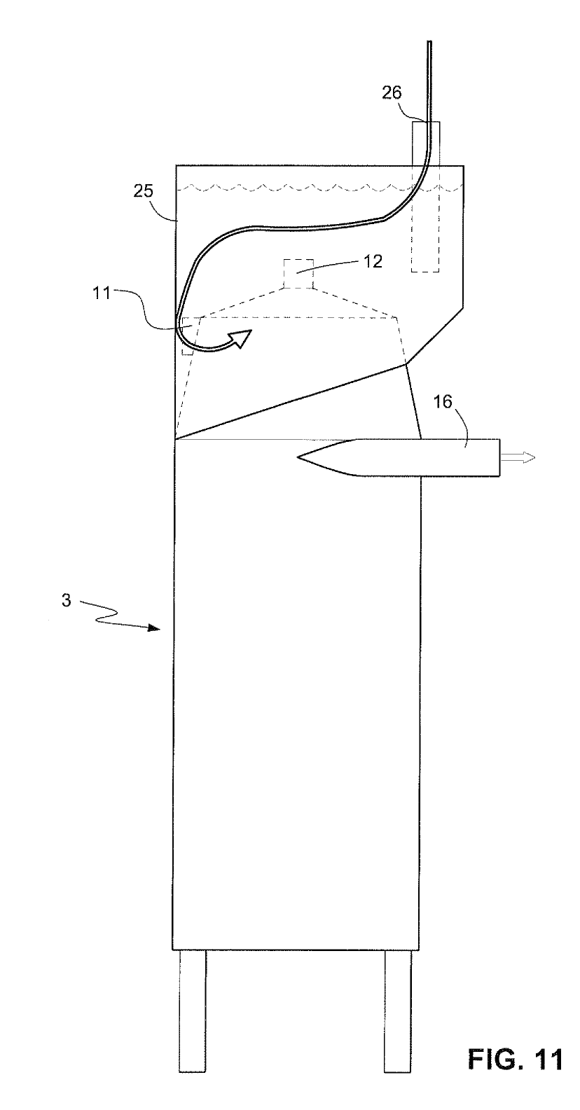

FIG. 1(b) is another non-limiting example CPB circuit in which gaseous emboli are removed. FIG. 1(b) is similar to FIG. 1(a) except that the ultrasound-assisted bubble trap is combined with the venous reservoir into one component 3b, while the configuration in FIG. 1(a) eliminates the venous reservoir from the main circuit loop. Both configurations are desirable in that they eliminate bubbles closer to their source which may have some clinical benefit in reduced inflammation due to platelet activation in the blood. The configuration in FIG. 1(b) may be more consistent with current practice in a CPB circuit than the configuration shown in FIG. 1(a) and therefore may be preferred. A more detailed non-limiting example of such a component 3b is shown in FIG. 11.

FIG. 1(c) is another non-limiting example CPB circuit in which gaseous emboli are removed. Here, the debubbling apparatus 3 is positioned on the arterial portion of the CPB circuit rather than on the venous side. Blood from the patient 1 is received at the venous reservoir 4 from which it is pumped by the circuit pump 5 to the oxygenator 7. The oxygenated blood from oxygenator 7 is then provided to bubble detector 2a, then to a debubbling apparatus implemented as a combined ultrasound-assisted bubble trap/arterial line filter 3c and bubble detector 2b before the blood is returned to the patient 1. This may be a desirable configuration because it removes gaseous from blood immediately prior to returning it the patient, and therefore, protects against small undetected leaks or other accidents that may occur downstream of the pump.

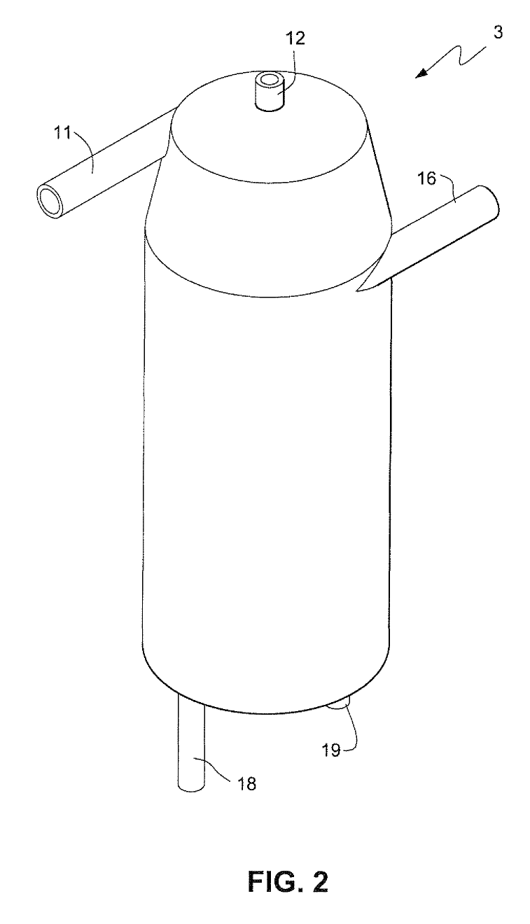

FIG. 2 is a front, perspective view of an ultrasound-assisted debubbling apparatus in accordance with a non-limiting example embodiment. The debubbling apparatus 3 is a generally cylindrically-shaped vessel that includes a fluid inlet port 11 for receiving a fluid to be debubbled, such as blood, a fluid outlet port 16 for outputting the debubbled fluid, and a bubble outlet port 12 for exhausting bubbles from the vessel that have been removed from the fluid. An ultrasonic transducer (not shown here), which transmits an ultrasonic beam through the received fluid in the vessel to move those bubbles toward the bubble outlet port 12 as will be illustrated and described further below, may generate heat that can be damaging the fluid and/or to the transducer itself. Accordingly, the other end of the vessel may include a cooling fluid inlet 18 and a cooling fluid outlet 19 for circulating cooling fluid in a portion of the vessel to cool the vessel and an ultrasonic transducer mounted in (or near) the vessel.

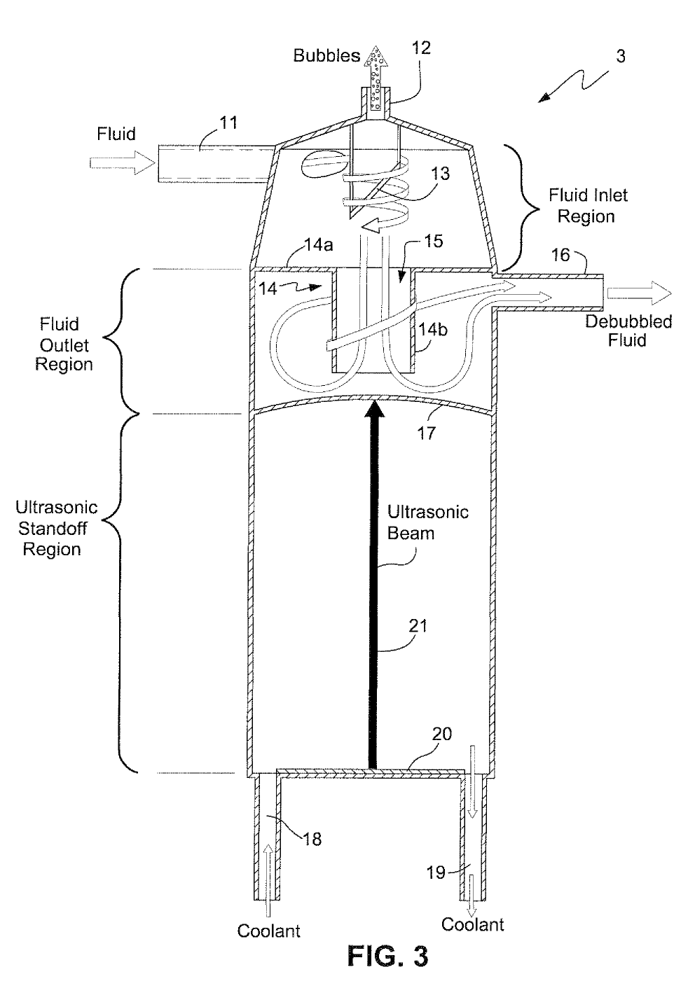

FIG. 3 is a cross-sectional view of the ultrasound-assisted debubbling apparatus in FIG. 2. The debubbling apparatus vessel is divided into three regions for ease of description: a fluid inlet region, a fluid outlet region, and an ultrasonic standoff region. Fluid enters the fluid inlet port 11 which preferably has a tangential orientation to the generally cylindrical shape of the vessel to encourage swirling flow of the blood inside the vessel as conceptually illustrated. Although the vessel is shown as being generally cylindrical, the vessel may be structured in other types of shapes. However, a cylindrically-shaped vessel is preferred because it also encourages swirling flow of the fluid which forces less dense particles such as air bubbles to the center of the vessel and coalesces them into larger bubbles that have sufficient buoyant force to rise to the top of the fluid inlet chamber where they may be removed via the bubble outlet port 12 as indicated. The fluid inlet chamber may be tapered as shown in FIG. 3 to direct the bubbles towards the bubble outlet port 12.

An ultrasonic transducer 20 is mounted at the other end of the vessel and generates an ultrasonic beam 21 that travels in a direction along a longitudinal axis of the vessel towards the fluid inlet region. The ultrasonic transducer 20 is preferably mounted within the vessel, but it may be mounted outside the vessel if desired. A non-limiting example of a suitable ultrasonic transducer is a lead zirconate titanate (PZT) crystal or another piezoelectric material that vibrates in response to an applied voltage. The transducer 20 is operated at a suitable power and frequency, e.g., by controller 9. A non-limiting example of power levels and frequency ranges for removing air bubbles from blood includes powers ranging from 1-190 W/cm² and frequencies ranging from 100 kHz to 10 MHz. Of course, these ranges are only examples and other frequencies and powers may be used. The ranges also depend on the application, the flow rate of the fluid, the viscosity of the fluid, and the size of the bubbles to be removed. The ultrasonic beam 21 carries momentum that is transferred to the air bubbles upon reflection or absorption of the ultrasonic beam which moves the bubbles towards the top of the fluid inlet region where they are withdrawn from the bubble outlet port 12.

The fluid inlet region is separated from a fluid outlet region by a barrier structure indicated generally at 14 that includes a first barrier portion 14a and a second barrier portion 14b. The barrier structure 14 may be made of biocompatible plastic such as polycarbonate but other materials may be used. The purpose of the barrier 14 is to block the bubbles from moving along with the fluid to the fluid outlet port 16 while at the same time still providing a path for the received fluid to reach the fluid outlet port 16. The first portion of the barrier 14a is a substantially horizontal surface with an opening 15 in the surface sufficiently aligned with the ultrasonic beam 21 so that at least a substantial portion of the ultrasonic beam energy reaches the fluid inlet region. The opening 15 in a preferred non-limiting example embodiment is circular so that the second barrier portion 14b is a cylinder substantially at a right angle to the first barrier portion 14a. The second barrier portion 14b confines the bubbles so that they are within the ultrasonic beam which maximizes the amount of power available to push the bubbles upward toward the bubble outlet port 12. Preferably, the dimensions of the opening 15 substantially match the cross-section of the ultrasonic beam 21 so that there is substantially uniform acoustic pressure across the opening 15 where the fluid passes from the fluid inlet region to the fluid outlet region. If the acoustic pressure is not uniform across the opening, bubbles may be able to pass through regions of the opening where the acoustic pressure is at a minimum.

Although the first and second barrier portions 14a and 14b are shown as perpendicular, they need not be and may be oriented in any position that transfers a substantial amount of the ultrasonic beam energy into the fluid inlet region while at the same time making it difficult for air bubbles to pass through the barrier opening into the fluid outlet region. Similarly, the shape of the barrier(s) and the opening need not as shown, but instead can be any suitable shape that transfers a substantial amount of the ultrasonic beam energy into the fluid inlet region while at the same time making it difficult for air bubbles to pass through the barrier opening into the fluid outlet region.

The fluid inlet region also includes an acoustic reflection element 13 that redirects the ultrasonic beam 21 away from the fluid outlet region as will be described in further detail below. Preferably, the reflection element 13 directs the ultrasonic beam 21 in such a way so as to maximize the amount of acoustic radiation force that is directed up toward the bubble removal port 12 in order to move bubbles in the blood in that direction.

A fluid barrier 17 made of acoustically transparent material separates the ultrasonic transducer 20 from the fluid. Non-limiting examples of acoustically transparent materials include polystyrene or mylar. As illustrated in FIG. 3, the region of the vessel between the ultrasonic transducer 20 and the fluid barrier 17 defines an ultrasonic stand-off region. Although not necessary, the fluid barrier 17 may be shaped to either focus or defocus the ultrasound beam 21 so that the beam profile substantially matches the dimensions of the opening 15 in the barrier 14. Example focusing properties of the fluid barrier 17 are described in further detail below.

One or more dimensions of the ultrasonic standoff region may be sized/shaped in order to increase or maximize the amount of acoustic energy transmitted into the fluid inlet region. One non-limiting example is to angle the sidewalls to serve as an acoustic collimator or by adjusting the distance between the ultrasonic transducer 20 and the acoustically transparent medium 17 so that the position of the fluid barrier 17 matches the near-field/far-field transition of the ultrasonic beam 21 where the sound wave is at a maximum. In high-frequency ultrasonic transducers, e.g., 1 MHz and up, this distance may be fairly large, e.g., on the order of 1 meter for a 1 cm beam width, which may have an attenuating effect on the ultrasonic beam 21. In this case, shortening the distance between the transducer and the acoustically transparent medium so that the fluid barrier is entirely within the near field may result in better performance.

To produce sufficient radiation force to drive microbubbles upward against the fluid flow, it may be desirable to drive the ultrasonic transducer at high powers that generate significant heat. In such a case, it would be desirable to cool the ultrasonic transducer 20. The ultrasonic standoff region receives cooling fluid through a coolant inlet 18 which is circulated in the ultrasonic standoff region and removed via a coolant outlet 19. Water is a non-limiting example coolant. Alternatively, externally applied coolant may be used to cool the walls of the standoff region. The ultrasonic standoff cooling fluid prevents the ultrasonic transducer 20 from overheating when operating at high powers required to force bubbles upwards at high flow rates. In CPB circuits, for example, the cooling water also prevents damage to blood from the heat generated by the ultrasonic transducer 20.

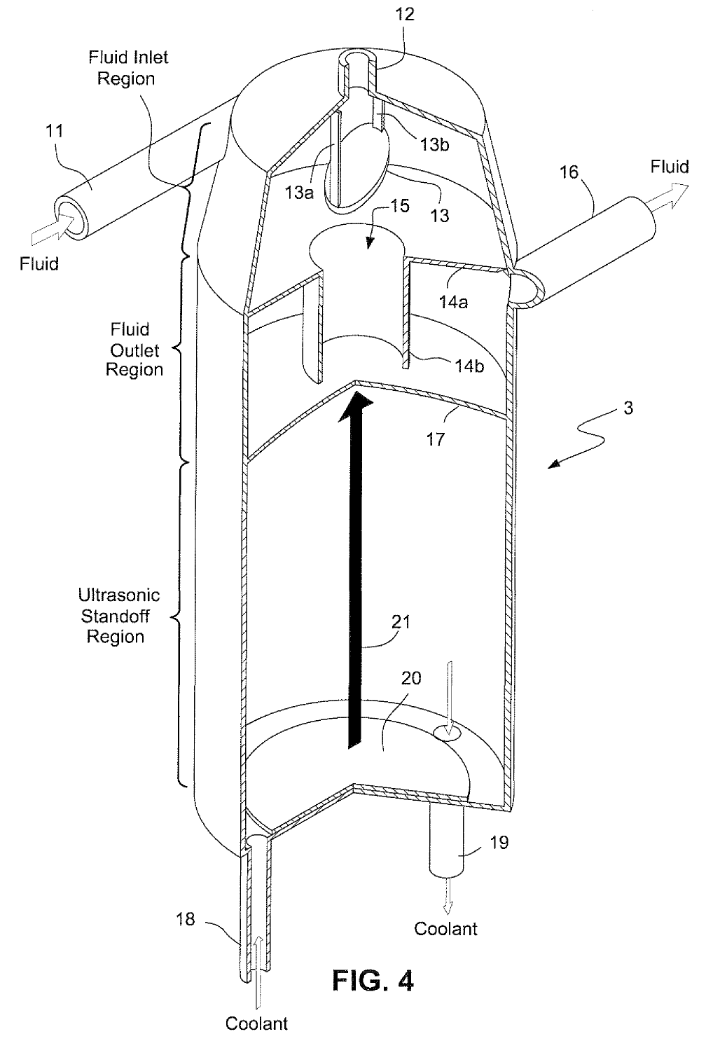

FIG. 4 is a three dimensional, perspective, cross-sectional view of the ultrasound-assisted debubbling apparatus of FIG. 2. This perspective cross-sectional view shows how the acoustic reflector 13 may be mounted inside the vessel using mounting members 13a and 13b. The perspective view also shows the barrier 14 with its first and second portions 14a and 14b which together form a cylindrical opening 15 that permits the fluid from the fluid inlet region to reach the fluid outlet region.

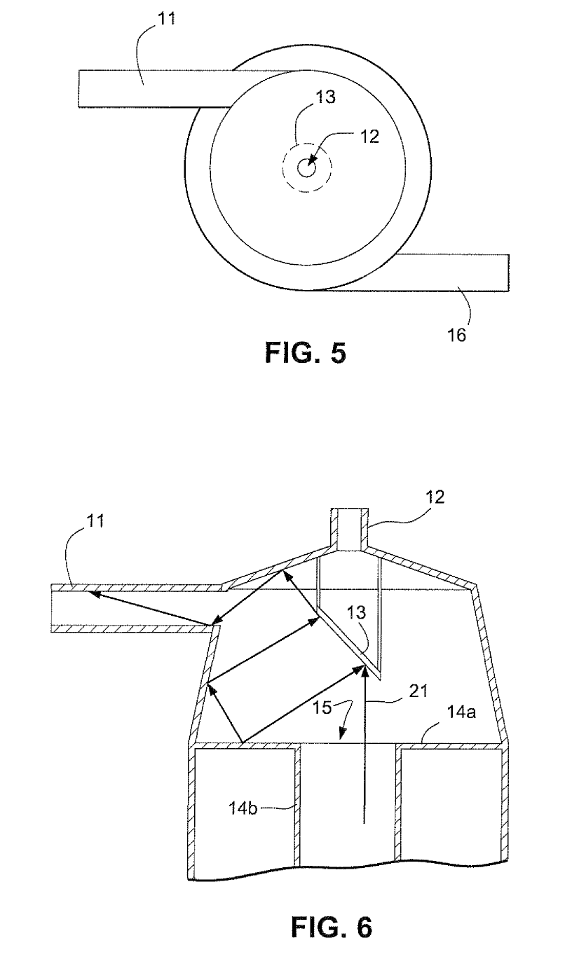

FIG. 5 is a top view of the debubbling apparatus in FIG. 2 and highlights the preferred, although not essential, tangential positioning of the fluid inlet port 11 and fluid outlet port 16 with respect to the vessel body to facilitate swirling flow of the fluid.

FIG. 6 is a partial cross-sectional drawing of the debubbling apparatus in FIG. 2 that illustrates how the acoustic reflector 13 redirects the ultrasonic beam 21 away from the opening 15 between the fluid inlet region and the fluid outlet region. The acoustic reflector 13 is angled in this non-limiting example so that the ultrasonic beam is reflected onto the first barrier portion 14a which reflects the beam toward the sidewalls of the vessel and then up to the top of the fluid inlet region, thereby pushing the air bubbles in the same upward direction toward the bubble outlet port 12.

After each reflection, some of the acoustic energy of the ultrasonic beam is transmitted into the reflecting material so that energy of the beam dissipates. Although after multiple reflections some acoustic energy may be directed downward through the opening 15, at that point, the energy of this multiply reflected beam will be substantially less than the energy of the incident ultrasonic beam coming up through the opening 15. In a preferred non-limiting implementation as shown, the reflector 13 is angled toward the fluid inlet port 11 so that the reflected acoustic energy hits bubbles immediately upon entering the apparatus so that the acoustic radiation force has more time to force bubbles upward toward the bubble outlet port.

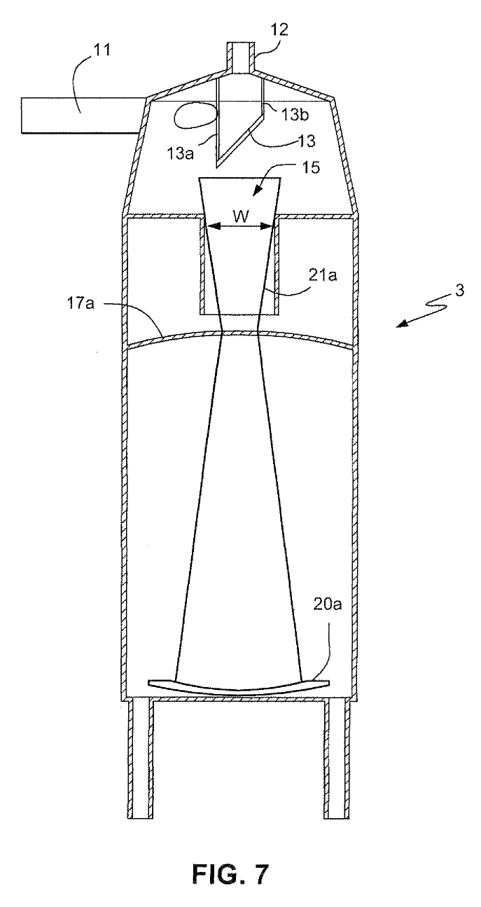

FIG. 7 is a cross-section of the ultrasound-assisted debubbling apparatus of FIG. 2 showing a non-limiting example embodiment with a curved ultrasonic transducer for shaping the ultrasonic beam. The ultrasonic transducer 20a is curved so as to focus the ultrasonic beam 21 towards the opening 15. As a result, the beam is more tightly collimated upon entering the fluid outlet region. In addition in this example embodiment, the fluid barrier 17a is shaped so as to defocus the beam. The beam diameter of the defocused beam increases to match the width W of the opening 15 between the fluid inlet and fluid outlet regions.

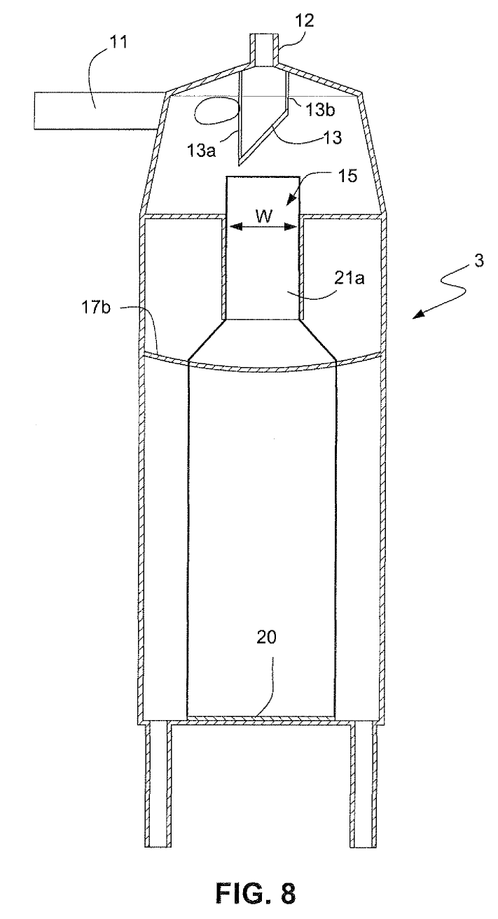

FIG. 8 is a cross-sectional view showing an alternative example embodiment of the debubbling apparatus in FIG. 2 showing a non-limiting example embodiment with a differently-shaped barrier separating an ultrasonic stand-off region from fluid regions in the vessel. The transducer 20 is not focused, and the fluid barrier 17b is shaped to focus the ultrasonic beam 21. Note in this example, the beam 21 is wider than the opening 15. The shape of the fluid barrier 17b is such that it focuses the acoustic beam 21 to substantially match the dimensions of the opening 15.

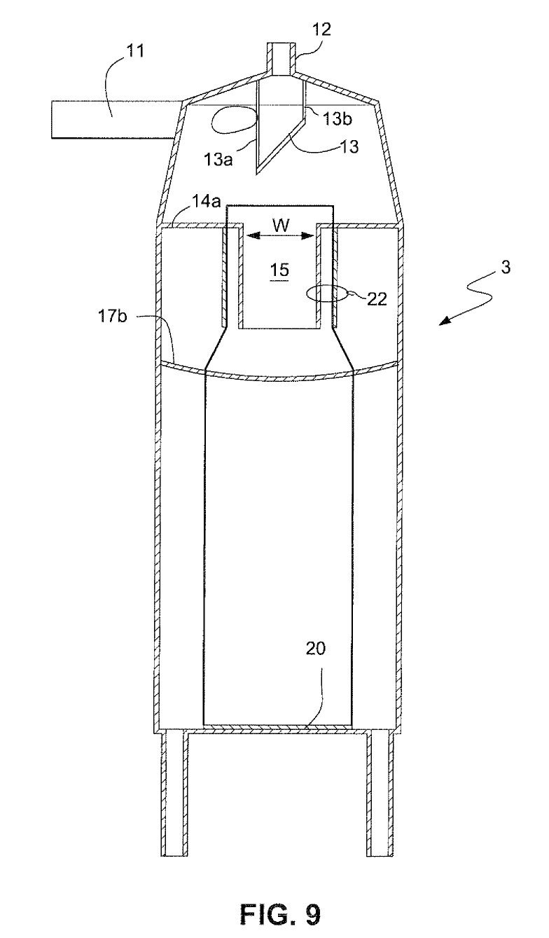

FIG. 9 is a cross-sectional view of the debubbling apparatus in FIG. 2 showing a non-limiting example embodiment with the barrier structure between the fluid inlet and fluid outlet ports having a concentrically-shaped portion. FIG. 9 is similar to FIG. 8 in that the ultrasound beam is wider than the opening 15. However, the second portion of the barrier 14, here shown at reference numeral 22, includes concentric cylinders. The fluid barrier 17b is shaped to focus the beam so that it matches the width of the external concentric cylinder of the concentric barrier 22. In this way, bubbles that pass down through the opening 15 must travel perpendicularly to the ultrasonic traveling wave contained within the ultrasonic beam 21 in order to pass to the fluid outlet port 16. Consequently, less ultrasonic force is required to push the bubbles up, and any bubbles with sufficient energy to enter the fluid outlet region can be trapped between the two concentric cylinders of barrier 22.

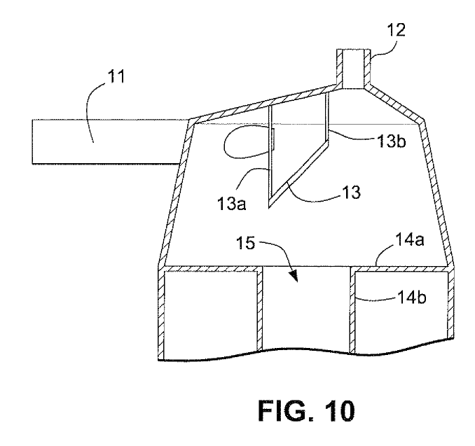

FIG. 10 is a partial cross-sectional view of the debubbling apparatus in FIG. 2 showing an alternative example embodiment where the bubble outlet port 12 is in a different location. Specifically, the bubble outlet port 12 is off-center from a central longitudinal axis of the debubbling vessel so that the outlet port is not directly above the acoustic reflector 13. As a result, the mounting members 13a and 13b will not block air from the reaching the bubble outlet port if they are constructed of a single cylindrical wall instead of two or more posts.

FIG. 11 is a side view of an alternative example embodiment for delivering fluid to the debubbling apparatus shown in FIG. 2. A container or bag 25 is used as a reservoir to receive and store fluid to be debubbled. The fluid to be debubbled is received from an inlet 26 and the pressure gradient within the CPB circuit pulls the fluid into the fluid inlet port 11. This configuration is an example for implementing the debubbling apparatus in the CPB circuit configuration shown in FIG. 1(b).

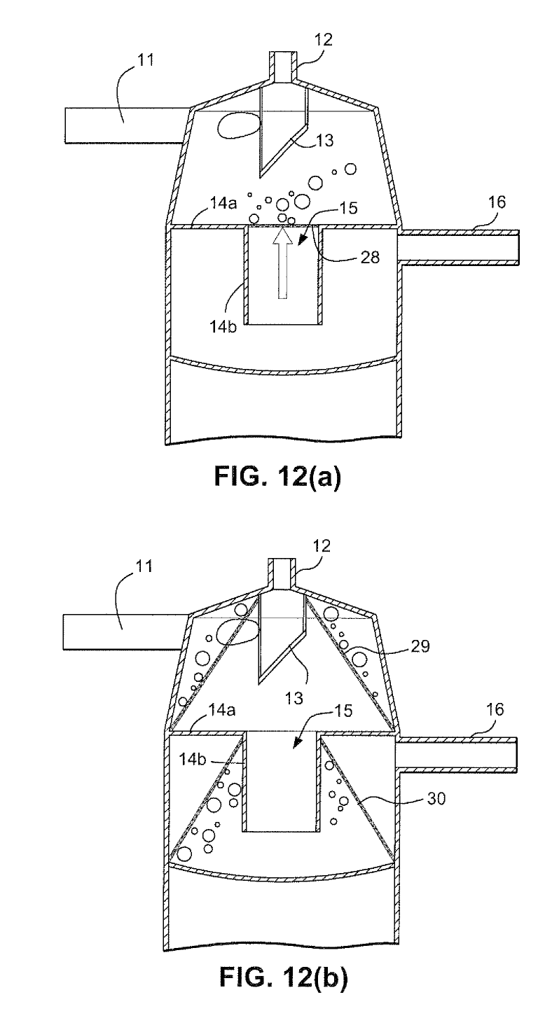

As is evident from the above description, the ultrasonic-assisted debubbling apparatus 3 includes multiple features that facilitate bubble removal from the fluid, which enhances the efficiency and reliability of the bubble removal process. Another bubble removal feature that may be used is one or more porous meshes to mechanically trap the bubbles or create barriers to the bubbles' movement within the vessel. FIG. 12a shows a non-limiting example embodiment where a porous mesh 28 is placed over the opening 15. The mesh 28 helps filter out bubbles from the fluid moving from the fluid inlet region to the fluid outlet region. In addition, the ultrasonic radiation force from the ultrasound beam can push the bubbles which are trapped in the porous mesh out of the mesh and back up toward the bubble outlet port 12. In other words, the ultrasound can “clear” trapped bubbles in the mesh.

Another alternative example mesh embodiment shown in FIG. 12b includes two conical mesh structures 29 and 30. The first conical mesh 29 structure is mounted in the fluid inlet region, and the second conical mesh structure 30 is mounted in the fluid outlet region. These meshes 29 and 30 are oriented at a substantial angle to the first barrier portion 14a. This substantial angle away from horizontal increases the surface area of the mesh, increasing the number of particles and bubbles that can be trapped without clogging the porous mesh and stopping flow.

By using ultrasonic radiation force in conjunction with mechanical features for debubbling a fluid (e.g., swirling flow, porous mesh filters, etc.), this technology removes bubbles from fluids more effectively than devices that just use ultrasound or mechanical features. In addition, the technology may be integrated into current CPB components and does not add fluid volume to the bypass circuit. In fact, by more effectively removing bubbles from fluid, it is possible to reduce the fluid volume of CPB circuit components, which results in less use of transfused blood during CPB surgery to maintain hematocrit and a reduced risk of systemic inflammation

Although various example embodiments have been shown and described in detail, the claims are not limited to any particular embodiment or example. None of the above description should be read as implying that any particular element, step, range, or function is essential such that it must be included in the claims scope. Reference to an element in the singular is not intended to mean “one and only one” unless explicitly so stated, but rather “one or more.” The scope of patented subject matter is defined only by the claims. The extent of legal protection is defined by the words recited in the allowed claims and their equivalents. All structural and functional equivalents to the elements of the above-described example embodiment that are known to those of ordinary skill in the art are expressly incorporated herein by reference and are intended to be encompassed by the present claims. Moreover, it is not necessary for a device or method to address each and every problem sought to be solved by the present invention for it to be encompassed by the present claims. No claim is intended to invoke paragraph 6 of 35 USC §112 unless the words “means for” or “step for” are used. Furthermore, no feature, component, or step in the present disclosure is intended to be dedicated to the public regardless of whether the feature, component, or step is explicitly recited in the claims.

1. A vessel for removing bubbles from a fluid comprising:
a fluid inlet port for receiving the fluid;
a bubble outlet port for removing bubbles in the fluid from the vessel;
an ultrasonic transducer mounted in the vessel and operable to transmit an ultrasonic beam through the received fluid to move bubbles in the fluid towards the bubble outlet port;
a fluid outlet port for outputting the fluid insonified by the ultrasonic beam; and
an ultrasonic reflector mounted near the bubble outlet port for reflecting the ultrasonic beam away from the fluid outlet port to reduce or prevent reflection of the ultrasonic beam off an interior surface in the vessel directed towards the fluid outlet port.

2. The vessel in claim 1, wherein the reflector is mounted to redirect reflected ultrasound beams away from the fluid outlet port.

3. The vessel in claim 2, wherein the reflector is mounted to reflect the ultrasonic beam so as to increase an amount of acoustic radiation force directed towards the bubble outlet port.

4. The vessel in claim 1, further comprising:
a barrier having a first barrier portion that separates the fluid inlet port and the fluid outlet port and an opening in the first barrier portion that permits the ultrasonic beam to radiate fluid passing from the fluid inlet port to the fluid outlet port.

5. The vessel in claim 4, wherein the opening is sized to at least substantially match a width of the ultrasonic beam.

6. The vessel in claim 4, wherein the reflector is positioned to reflect the ultrasonic beam away from the opening.

7. The vessel in claim 4, wherein the barrier includes a second barrier portion at a sufficient angle to the first barrier portion to create the opening, the second barrier portion extending past the fluid outlet port.

8. The vessel in claim 7, wherein the first barrier portion is substantially perpendicular to a sidewall of the vessel, and wherein the first barrier portion and the second barrier portion are substantially perpendicular.

9. The vessel in claim 8, wherein the opening is circular and the second barrier portion is cylindrically-shaped.

10. The vessel in claim 9, wherein the second barrier portion includes concentric cylindrically-shaped surfaces.

11. The vessel in claim 4, further comprising:
an acoustically transparent material separating the ultrasonic transducer from the fluid inlet port and the fluid outlet port.

12. The vessel in claim 11, further comprising:
a cooling fluid inlet for inputting cooling fluid to remove heat from the vessel caused by the ultrasonic transducer, and
a cooling fluid outlet for removing the cooling fluid from the vessel,
wherein the acoustically transparent material prevents the cooling fluid from contacting the received fluid.

13. The vessel in claim 11, wherein the acoustically transparent material is shaped to adjust the ultrasound beam so that a profile of the ultrasound beam approximates the dimensions of the opening in the barrier.

14. The vessel in claim 11, wherein an ultrasonic standoff region is formed in the vessel between the acoustically transparent material and the ultrasonic transducer, and wherein a length of the ultrasonic standoff region substantially matches a near-field/far-field transition of the ultrasonic beam where the ultrasonic wave is at a maximum amplitude.

15. The vessel in claim 1, wherein the ultrasound beam, the opening in the barrier, and the bubble outlet port are substantially aligned along a same axis.

16. The vessel in claim 15, wherein the acoustic reflector is substantially aligned along the same axis.

17. The vessel in claim 15, wherein the acoustic reflector is offset from and not aligned with the same axis.

18. The vessel in claim 1, wherein the ultrasonic transducer is shaped to focus the energy of the ultrasonic beam through the opening.

19. The vessel in claim 1, wherein the vessel is cylindrically-shaped, and wherein the fluid inlet port and the fluid outlet port are substantially tangential to a cylindrical surface of the vessel to produce a swirling flow of the received fluid in the vessel that forces bubbles to the center of the vessel in line with the opening and coalesces smaller ones of the bubbles into larger bubbles.

20. The vessel in claim 1, wherein the bubble outlet is located at or near a highest point of the vessel when the vessel is mounted for operation.

21. The vessel in claim 1, further comprising:
a porous mesh positioned in a direction that is substantially parallel to the first barrier portion and covers the opening,
wherein the porous mesh mechanically traps bubbles and other foreign particles larger than a pore size of the porous mesh and the ultrasonic beam forces the bubbles towards the bubble outlet port.

22. The vessel in claim 1, further comprising:
a porous mesh positioned in a direction having a substantial angle with the first barrier portion between the fluid inlet port and the opening and between the opening and the fluid outlet port,
wherein the porous mesh mechanically traps bubbles and other foreign particles larger than a pore size of the porous mesh.

23. A system for removing gaseous emboli from blood, comprising:
a blood circuit receiving blood from a patient;
a pump coupled to the blood circuit for pumping the blood through the blood circuit;
a vessel coupled to the blood circuit for removing gaseous emboli from blood including:
a blood inlet port for receiving the blood;
an emboli outlet port for removing gaseous emboli in the blood from the vessel;
an ultrasonic transducer mounted in the vessel and operable to transmit an ultrasonic beam through the received fluid to move gaseous emboli in the fluid towards the gaseous emboli outlet port;
a blood outlet port for outputting the blood insonified by the ultrasonic beam; and
an ultrasonic reflector mounted near the gaseous emboli outlet port for reflecting the ultrasonic beam away from the blood outlet port to reduce or prevent reflection of the ultrasonic beam off an interior surface in the vessel directed towards the blood outlet port; and
a controller for controlling the ultrasonic transducer and the pump.

24. The system in claim 23, wherein the blood circuit further comprises a sensor for sensing gaseous emboli in the blood entering the vessel and providing sensor information to the controller for use by the controller in controlling operation of the ultrasonic transducer.

25. The system in claim 23, wherein the blood circuit further comprises a sensor for sensing gaseous emboli in the blood exiting the vessel to detect when gaseous emboli still remains in the blood.

26. The system in claim 23, wherein the vessel is provided in one or more of the following components included in the blood circuit: a venous reservoir, an arterial line filter, or a bubble trap.

27. The system in claim 23, wherein the reflector is mounted to reflect the ultrasonic beam away from the gaseous emboli outlet port and to increase an amount of acoustic radiation force directed upwards towards the gaseous emboli outlet port.

28. The system in claim 23, wherein the vessel further comprises:
a barrier having a first barrier portion that separates the blood inlet port and the blood outlet port and an opening in the first that permits the ultrasonic beam to radiate blood received from the blood inlet port and received blood from the blood inlet port to reach the blood outlet port.

29. The system in claim 28, wherein the opening is sized to at least substantially match a width of the ultrasonic beam, and wherein the reflector is positioned to reflect the ultrasonic beam away from the opening.

30. The system in claim 28, wherein the barrier includes a second barrier portion at a sufficient angle to the first barrier portion to create the opening, the second barrier portion extending past the blood outlet port.

31. The system in claim 28, wherein the vessel further includes:
a cooling fluid inlet for inputting cooling fluid to remove heat from the vessel caused by the ultrasonic transducer,
a cooling fluid outlet for removing the cooling fluid from the vessel, and
an acoustically transparent material separating the cooling fluid from the blood;
wherein the acoustically transparent material the permits transmission of ultrasonic energy into the received blood while preventing the cooling fluid from contacting the received blood.

32. A method for debubbling a liquid comprising:
introducing the liquid to a vessel through a fluid inlet;
causing the liquid to flow through the vessel in a spiral path toward a first outlet;
operating an ultrasonic transducer within the vessel to transmit an ultrasonic beam along a longitudinal axis the vessel toward the spiral path and toward a second outlet;
reflecting the ultrasonic beam within the vessel away from the blood outlet port to reduce or prevent reflection of the ultrasonic beam off an interior surface in the vessel directed towards the first outlet;
withdrawing a stream of insonified liquid through the first outlet; and
withdrawing a stream of liquid containing entrained air bubbles through the second outlet.

33. The method in claim 32, further comprising reflecting the ultrasonic beam away from the first outlet to increase an amount of acoustic radiation force directed upwards towards the second outlet.

34. The method in claim 32, further comprising:
separating the inlet and the first outlet using a barrier having a first barrier portion,
wherein an opening in the first barrier portion permits the ultrasonic beam to radiate fluid received from the inlet before the received fluid reaches the first outlet.

35. The method in claim 32, further comprising:
separating the ultrasonic transducer from the inlet and the first outlet with an acoustically transparent material.

36. The method in claim 35, further comprising:
inputting cooling fluid via a cooling fluid inlet to remove heat from the vessel caused by the ultrasonic transducer, and
removing the cooling fluid from the vessel via a cooling fluid outlet,
wherein the acoustically transparent material prevents the cooling fluid from contacting the received fluid.

37. The method in claim 35, further comprising:
shaping the ultrasound beam so that a profile of the ultrasound beam approximates the dimensions of the opening in the barrier.
